(19) United States
(12) Patent Application Publication
(10) Pub. No.: US 2013/0096356 A1
(43) Pub. Date: Apr. 18, 2013

(54) METHODS AND APPARATUSES FOR THE ISOMERIZATION AND DEISOHEXANIZING OF HYDROCARBON FEEDS

(71) Applicants: Laxmikant V. Bharuka, Gurgaon (IN); Heidi C. Fraser, Lombard, IL (US); David James Shecterle, Arlington Heights, IL (US)

(72) Inventors: Laxmikant V. Bharuka, Gurgaon (IN); Heidi C. Fraser, Lombard, IL (US); David James Shecterle, Arlington Heights, IL (US)

(73) Assignee: UOP LLC, Des Plaines, IL (US)

(21) Appl. No.: 13/273,609

(22) Filed: Oct. 14, 2011

(51) Int. Cl. C07C 5/22; B01J 8/00

(52) U.S. Cl. 585/317; 422/625; 422/187

(57) ABSTRACT

Methods and apparatuses for the isomerization and deisohexanizing of a feed are provided. In a method for the isomerization of a feed in an isomerization zone to form an isomerized stream and the deisohexanizing of the isomerized stream in a deisohexanizer zone, heat is exchanged between the isomerization zone and the deisohexanizer zone to raise the temperature of the feed and to reduce the temperature of the deisohexanizer sidecut stream.

### TECHNICAL FIELD

This document generally relates to methods and apparatuses for the isomerization and deisohexanizing of hydrocarbon feeds, and more particularly relates to such methods and apparatuses that provide enhanced heat recovery.

### BACKGROUND

Isomerization and deisohexanizing processing of hydrocarbons is well developed and widely practiced in the petrochemical and petroleum refining industries. One constant concern for petrochemical and petroleum refiners is the utility consumption of isomerization and deisohexanizing processing units. One method of reducing utility consumption in isomerization processing is to use a heat exchange between hot streams with excess heat and cooler streams in need of energy. For instance, the standard process flow in a typical isomerization process is to heat the feed stream by indirect heat exchange against the effluent of the isomerization zone.

While current methods are able to utilize heat energy from effluent isomerization streams to preheat feedstock, the methods typically still require large amounts of utility consumption. For instance, current methods typically utilize additional heating of feedstock by passing the feedstock stream through a steam heater or a similar available source of high temperature heat. Due to the large scale of the processing, a nominal improvement in energy efficiency can significantly reduce utility consumption.

Accordingly, it is desirable to provide methods and apparatuses for the isomerization and deisohexanizing of hydrocarbon feeds that provide enhanced heat recovery. In addition, it is desirable to provide methods and apparatuses for the isomerization and deisohexanizing of hydrocarbon feeds that exchange heat between the feed and a sidecut from the deisohexanizer unit. Furthermore, other desirable features and characteristics will become apparent from the subsequent detailed description and the appended claims, taken in conjunction with the accompanying drawings and this background.

### BRIEF SUMMARY

Methods and apparatuses for the isomerization and deisohexanizing of hydrocarbon feeds are provided. In accordance with an exemplary embodiment, a method for the isomerization of a feed in an isomerization zone to form an isomerized stream and the deisohexanizing of the isomerized stream in a deisohexanizer zone includes exchanging heat between the isomerization zone and the deisohexanizer zone to raise the temperature of the feed.

In accordance with another exemplary embodiment, a method for isomerization and deisohexanizing of a hydrocarbon feed includes isomerizing the hydrocarbon feed in a lead isomerization reactor to form a first isomerized stream. The first isomerized stream is isomerized in a lag isomerization reactor to form a second isomerized stream. The second isomerized stream is deisohexanized in a deisohexanizer. The method provides for drawing a sidecut stream from the deisohexanizer. Heat is exchanged between the hydrocarbon feed and the sidecut stream from the deisohexanizer to raise the temperature of the hydrocarbon feed. Further, heat is exchanged between the hydrocarbon feed and the second isomerized stream after heat is exchanged between the hydrocarbon feed and the sidecut stream. Also, heat is exchanged between the hydrocarbon feed and the first isomerized stream after heat is exchanged between the hydrocarbon feed and the second isomerized stream.

Another exemplary embodiment provides an apparatus for the isomerization and deisohexanizing of a hydrocarbon feed. The apparatus includes an isomerization zone including an isomerization unit configured to isomerize the hydrocarbon feed to form an isomerized stream. Further, the apparatus includes a deisohexanizer zone located downstream of the isomerization zone and configured to deisohexanize the isomerized stream. Also, the apparatus includes a heat exchanger coupled between the isomerization zone and deisohexanizer zone and configured to transfer heat to the feed upstream of the isomerization unit.

### BRIEF DESCRIPTION OF THE DRAWINGS

Exemplary embodiments will hereinafter be described in conjunction with the following drawing figure, wherein:

FIG. 1 is simplified schematic representation of an isomerization and deisohexanizer apparatus arranged in accordance with an exemplary embodiment herein.

### DETAILED DESCRIPTION

The following detailed description is merely exemplary in nature and is not intended to limit the isomerization and deisohexanizing methods and apparatuses described. Furthermore, there is no intention to be bound by any theory presented in the preceding background or the following detailed description. Also, additional components, loops, and processes may be included in the apparatus but are not described herein for purposes of clarity. Stream compositions presented herein are merely illustrative of an embodiment and are not intended to limit the methods and apparatuses in any way.

The UOP Penex™ process is a continuous catalytic process used in the refining of crude oil. The process isomerizes hydrocarbon feeds into higher octane, branched molecules. For example, a hydrocarbon feed such as light naphtha, which typically comprises C₄-C₇ paraffins and C₅-C₇ cyclic hydrocarbons, and often primarily comprises C₅ and C₆ paraffins, may be isomerized into higher-octane, branched C₅/C₆ molecules. The process typically uses reactors with high activity chlorinated alumina-type platinum catalysts. A single pass of feedstock with an octane rating of 50-60 through such a reactor typically produces an end product rated at 82-86. To obtain a higher octane rating, the feedstock may be subsequently passed through a deisohexanizer (DIH) unit. After deisohexanizing, the end product typically has an octane rating of 87-90.5.

Methods and apparatuses for isomerization and deisohexanizing of hydrocarbon feeds are contemplated herein. The methods and apparatuses achieve enhanced heat recovery through heat exchange between deisohexanizer and isomerization stages. To that end, heat is exchanged between a sidecut from the deisohexanizer zone and the feed into the isomerization unit. As a result, heat energy is efficiently transferred between the deisohexanizer zone and the isomerization zone within the apparatus, and the need for additional heat input from outside the apparatus is reduced.

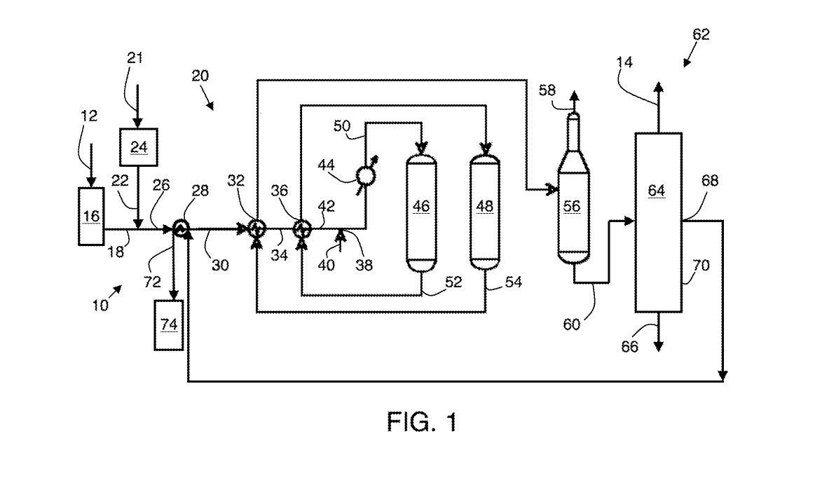

As shown in FIG. 1, the exemplary isomerization and deisohexanizer apparatus 10 refines a hydrocarbon feed 12 to create a high octane product 14. In an exemplary embodiment, the feed 12 may be primarily comprised of C₅ and C₆ paraffins and include some C₇ paraffins. Certain feeds may include between 1% and 5%, 10%, or even more than 10%, C₇ paraffins. The processing of hydrocarbon feeds 12 having other compositions is also contemplated for the apparatus 10. The feed 12 is received by charge pump 16 and is then fed through line 18 toward an isomerization zone 20. As shown, the output of the charge pump 16 may be combined with make up hydrogen 21. Preferably the make up hydrogen 21 is delivered via line 22 after having been dried by dryer 24 to eliminate any water or sulfur content therein to form a combined feed in line 26. The combined feed in line 26 is then heated by a first indirect heat exchanger 28. Line 30 delivers the output of the first indirect heat exchanger 28 to a second indirect heat exchanger 32 for further heating. The output of the second indirect heat exchanger 32 then flows through line 34 for heating by a third indirect heat exchanger 36. An injector 38 adds a chloride source 40, such as perchloroethylene, to the heated output of the third indirect heat exchanger 36 in line 42. The chlorided feed in line 42 is then heated by a charge heater 44 or the like.

As shown, the isomerization zone 20 includes an isomerization unit comprised of a lead isomerization reactor 46 and a lag isomerization reactor 48. While two reactors are shown, in certain embodiments there may be either one or three or more isomerization reactors. Reactors 46 and 48 may be substantially identical, with “lead” and “lag” only referring to their positioning in relation to fluid flow in the apparatus 10. In certain embodiments, the catalyst used in the isomerization zone 20 is distributed equally between the reactors 46 and 48. In other embodiments, there may be differing catalyst distributions. The use of multiple reactors 46 and 48 facilitates a variation in the operating conditions between the two reaction zones to enhance isoparaffin production and improve cyclic hydrocarbon conversion. In this manner, the lead reactor 46 can operate at higher temperature conditions that favor ring opening but performs only a portion of the normal to isoparaffin conversion. The heat exchangers upstream of the lead isomerization reactor 46, facilitate the use of higher temperatures in the lead isomerization reactor 46. Once cyclic hydrocarbon rings have been opened by initial contact with the catalyst, the lag reactor 48 may operate at temperature conditions that are more favorable for isoparaffin equilibrium.

In FIG. 1, line 50 delivers the output from the charge heater 44 to the lead reactor 46 where isomerization at higher temperatures occurs, producing a hot isomerized stream 52. Isomerized stream 52 is directed to the third indirect heat exchanger 36 where it heats the output of the second indirect heat exchanger 32 carried by line 34. Then, isomerized stream 52 is passed to lag reactor 48 where additional isomerization over the catalysts therein occurs at lower temperatures. As a result of the additional isomerization, a cooler isomerized stream 54 is produced. Isomerized stream 54 is passed through the second indirect heat exchanger 32 and heats the output of the first indirect heat exchanger 28 carried by line 30.

After passing through the second indirect heat exchanger 32, isomerized stream 54 exits the isomerization zone 20 and enters a fractionating column or stabilizer 56. Stabilizer 56 separates an overhead offgas product 58 typically containing HCL, hydrogen, and light hydrocarbons such as byproduct methane, ethane, propane and butane gases. Offgas 58 is scrubbed to remove HCL and then may be routed to a central gas processing plant for removal and recovery of hydrogen, propane and butane. The residual gas after such processing may become part of the refinery's fuel gas system. In FIG. 1, the stabilizer 56 forms a bottoms product 60 that includes liquid isomerate to be fed to a deisohexanizer zone 62.

In the deisohexanizer zone 62, a deisohexanizer unit 64 deisohexanizes the bottoms product 60 and creates high octane isomerate 14 and a bottoms product 66. As shown, the deisohexanizer unit 64 includes an outlet 68 for a sidecut stream 70. In an exemplary embodiment, the sidecut stream 70 is comprised primarily of normal hexane and monomethylpentanes, particularly normal hexane, 2-methylpentane and 3-methylpentane. The exemplary sidecut stream 70 may also contain cyclohexane, some dimethylbutanes, and some heavies. Sidecut streams 70 having other compositions are contemplated herein, and are envisioned as a result of differing feedstocks and differing processing. In FIG. 1, the sidecut stream 70 passes through the first indirect heat exchanger 28 to heat the combined feed in line 26 upstream of the second indirect heat exchanger 32. The sidecut stream 70 then exits the isomerization zone 20 via line 72. As a result of the flows into the first indirect heat exchanger 28, heat is exchanged between the deisohexanizer zone 62 and the isomerization zone 20 upstream of the isomerization unit 46 and 48. After the heat exchange, the sidecut stream 70 may be delivered to a cooler 74 to be cooled further, and after cooling, the sidecut stream 70 may be fed into the feed 12.

In an exemplary embodiment of the apparatus 10, the temperature of the sidecut stream 70 is about 110° C. when exiting the deisohexanizer unit 64. After heat exchange at the first indirect heat exchanger 28, the temperature of the sidecut stream 70 is about 66° C.-68° C. At the first indirect heat exchanger 28, the temperature of the combined feed from line 26 is raised from about 43° C. to about 64° C.

At the second indirect heat exchanger 32, the fluid in line 30 is heated from about 64° C. to about 115° C., while the isomerized stream 54 is cooled from about 174° C. to about 135° C. At the third indirect heat exchanger 36, the fluid from line 34 is heated from about 115° C. to about 175° C., while the isomerized stream 52 is cooled from about 204° C. to about 146° C. As a result of the increased temperature of the output from the third indirect heat exchanger 36 in line 42, less energy is needed from the charge heater 44 before the isomerization reaction.

Accordingly, apparatuses and methods for the isomerization and deisohexanizing of hydrocarbon feeds have been provided. While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the isomerization and deisohexanizer apparatuses or methods in any way. Rather, the foregoing detailed description will provide those skilled in the art with a convenient road map for implementing an exemplary embodiment, it being understood that various changes may be made in the function and arrangement of elements described in an exemplary embodiment without departing from the scope as set forth in the appended claims and their legal equivalents.

What is claimed is:

1. A method for the isomerization of a feed in an isomerization zone to form an isomerized stream and the deisohexanizing of the isomerized stream in a deisohexanizer zone, the method comprising:
exchanging heat between the isomerization zone and the deisohexanizer zone to raise the temperature of the feed.

2. The method of claim 1 further comprising combining hydrogen with the feed before the isomerization of the feed.

3. The method of claim 2 further comprising injecting a chloride source into the feed before the isomerization of the feed.

4. The method of claim 3 further comprising heating the feed with a charge heater after injecting the chloride source into the feed.

5. The method of claim 4 further comprising delivering the isomerized stream to a stabilizer to remove light hydrocarbons from the isomerized stream before deisohexanizing.

6. The method of claim 1 wherein isomerization is performed in a lead reactor and a lag reactor, wherein the feed is reacted in the lead reactor to form a first isomerized stream, and wherein the first isomerized stream is reacted in the lag reactor to form a second isomerized stream.

7. The method of claim 6 wherein the exchanging heat step is accomplished by exchanging heat from a sidecut stream from the deisohexanizer zone with the feed, and further comprising exchanging heat between the feed and the second isomerized stream after exchanging heat between the feed and the sidecut stream.

8. The method of claim 7 further comprising exchanging heat between the feed and the first isomerized stream after exchanging heat between the feed and the second isomerized stream.

9. The method of claim 8 further comprising injecting a chloride source into the feed after exchanging heat between the feed and the first isomerized stream.

10. The method of claim 9 further comprising heating the feed with a charge heater after injecting the chloride source into the feed.

11. The method of claim 10 further comprising delivering the isomerized stream to a stabilizer to remove light hydrocarbons from the isomerized stream before deisohexanizing.

12. The method of claim 1 wherein heat from a sidecut stream from the deisohexanizer zone is exchanged with the feed, wherein the sidecut stream includes normal hexane and methylpentanes, and wherein the method further comprises cooling the sidecut stream after exchanging heat between the feed with the sidecut stream.

13. A method for isomerization and deisohexanizing of a hydrocarbon feed comprising:
isomerizing the hydrocarbon feed in a lead isomerization reactor to form a first isomerized stream;
isomerizing the first isomerized stream in a lag isomerization reactor to form a second isomerized stream;
deisohexanizing the second isomerized stream in a deisohexanizer;
drawing a sidecut stream from the deisohexanizer;
exchanging heat between the hydrocarbon feed and the sidecut stream from the deisohexanizer to raise the temperature of the hydrocarbon feed;
exchanging heat between the hydrocarbon feed and the second isomerized stream after exchanging heat between the hydrocarbon feed and the sidecut stream; and
exchanging heat between the hydrocarbon feed and the first isomerized stream after exchanging heat between the hydrocarbon feed and the second isomerized stream.

14. The method of claim 13 further comprising combining hydrogen with the hydrocarbon feed.

15. The method of claim 13 further comprising injecting a chloride source into the hydrocarbon feed after exchanging heat between the hydrocarbon feed and the first isomerized stream.

16. The method of claim 15 further comprising heating the hydrocarbon feed with a charge heater after injecting the chloride source into the hydrocarbon feed.

17. The method of claim 16 further comprising delivering the second isomerized stream to a stabilizer to remove light hydrocarbons from the second isomerized stream before deisohexanizing the second isomerized stream.

18. The method of claim 17 wherein the sidecut stream comprises normal hexane and methylpentane, and further comprising delivering the sidecut stream to a cooler after exchanging heat between the sidecut stream and the hydrocarbon feed.

19. An apparatus for the isomerization and deisohexanizing of a hydrocarbon feed comprising:
an isomerization zone including an isomerization unit configured to isomerize the hydrocarbon feed to form an isomerized stream;
a deisohexanizer zone located downstream of the isomerization zone and configured to deisohexanize the isomerized stream; and
a heat exchanger coupled between the isomerization zone and deisohexanizer zone and configured to transfer heat to the feed upstream of the isomerization unit.

20. The apparatus of claim 19 wherein the isomerization unit includes a lead isomerization reactor configured to isomerize the hydrocarbon feed to form a first isomerized stream, and a lag isomerization reactor located downstream of the lead isomerization reactor and configured to receive and isomerize the first isomerized stream to form a second isomerized stream;
wherein the deisohexanizer zone includes a deisohexanizer unit located downstream of the lag isomerization reactor and configured to deisohexanize the second isomerized stream, wherein the deisohexanizer unit includes an outlet for a sidecut stream;
wherein the heat exchanger is configured to transfer heat from the sidecut stream to the hydrocarbon feed upstream of the lead isomerization reactor; and
wherein the apparatus further comprises:
a second heat exchanger configured to transfer heat from the second isomerized stream to the hydrocarbon feed upstream of the lead isomerization reactor;
a third heat exchanger configured to transfer heat from the first isomerized stream to the hydrocarbon feed upstream of the lead isomerization reactor;
an injector located upstream of the lead isomerization reactor and configured to inject a chloride source into the hydrocarbon feed;
a charge heater located upstream of the lead isomerization reactor and configured to heat the hydrocarbon feed; and
a stabilizer located downstream of the lag reactor and upstream of the deisohexanizer and configured to remove light hydrocarbons from the second isomerized stream.
